(19) United States
(12) Patent Application Publication
(10) Pub. No.: US 2003/0092180 A1
(43) Pub. Date: May 15, 2003

(54) Inhibition of gene expression by delivery of small interfering RNA to post-embryonic animal cells in vivo

(76) Inventors: David Lewis, Madison, WI (US); Hans Herweijer, Madison, WI (US); James E. Hagstrom, Madison, WI (US); Aaron G. Loomis, Prairie du Sac, WI (US); Jon A. Wolff, Madison, WI (US)

Correspondence Address: Mark K. Johnson, PO Box 510644, New Berlin, WI, 53151-0644, US

(21) Appl. No.: 10/012,804

(22) Filed: Nov. 6, 2001

Related U.S. Application Data
(60) Provisional application No. 60/315,394, filed on Aug. 27, 2001.
(60) Provisional application No. 60/324,155, filed on Sep. 20, 2001.

(51) Int. Cl. A61K 48/00; C12N 15/85

(52) U.S. Cl. 435/455; 514/44; 435/375

(57) ABSTRACT

A process is provided to deliver small interfering RNA to cells in vivo for the purpose of inhibiting gene expression in that cell. The small interfering RNA is less than 50 base-pairs in length. This process is practiced on post-embryonic animals. Inhibition is sequence-specific and relies on sequence identity of the small interfering RNA and the target nucleic acid molecule.

This Patent Application is related to pending U.S. patent applications No. 60/315,394 filed Aug. 27, 2001 and No. 60/324,155 filed Sep. 20, 2001.

### FIELD

The present invention generally relates to inhibiting gene expression. Specifically, it relates to inhibiting gene expression by delivery of small interfering RNAs (siRNAs) to post-embryonic animals.

### BACKGROUND

RNA interference (RNAi) describes the phenomenon whereby the presence of double-stranded RNA (dsRNA) of sequence that is identical or highly similar to a target gene results in the degradation of messenger RNA (mRNA) transcribed from that targeted gene (Sharp 2001). RNAi is likely mediated by siRNAs of approximately 21-25 nucleotides in length which are generated from the input dsRNAs (Hammond, Bernstein et al. 2000; Parrish, Fleenor et al. 2000; Yang, Lu et al. 2000; Zamore, Tuschl et al. 2000; Bernstein, Caudy et al. 2001).

The ability to specifically knock-down expression of a target gene by RNAi has obvious benefits. For example, RNAi could be used to generate animals that mimic true genetic “knockout” animals to study gene function. In addition, many diseases arise from the abnormal expression of a particular gene or group of genes. RNAi could be used to inhibit the expression of the genes and therefore alleviate symptoms of or cure the disease. For example, genes contributing to a cancerous state could be inhibited. In addition, viral genes could be inhibited, as well as mutant genes causing dominant genetic diseases such as myotonic dystrophy. Inhibiting such genes as cyclooxygenase or cytokines could also treat inflammatory diseases such as arthritis. Nervous system disorders could also be treated. Examples of targeted organs would include the liver, pancreas, spleen, skin, brain, prostrate, heart etc.

The introduction of dsRNA into mammalian cells is known to induce an interferon response which leads to a general block in protein synthesis and leads to cell both by both nonapoptotic and apoptotic pathways (Clemens and Elia 1997). In fact, studies performed using mammalian cells in culture indicate that introduction of long, double-stranded RNA does not lead to specific inhibition of expression of the target gene (Tuschl, Zamore et al. 1999; Caplen, Fleenor et al. 2000). A major component of the interferon response is the dsRNA-dependent protein kinase, PKR that phosphorylates and inactivates the elongation factor eIF2a. In addition, dsRNA induces the synthesis of 2′-5′ polyadenylic acid leading to the activation of the non-sequence specific RNase, RNaseL) (Player and Torrence 1998). PKR is not activated by dsRNA of less than 30 base pairs in length (Minks, West et al. 1979; Manche, Green et al. 1992).

In mammals, it has previously been demonstrated that long double-stranded RNA can be used to inhibit target gene expression in mouse oocytes and embryos (Svoboda, Stein et al. 2000; Wianny and Zemicka-Goetz 2000). It is likely that the interferon response pathway is not present in these cells at this early developmental stage. Recently, it has been shown that siRNA<30 bp can be used to induce RNAi in mammalian cells in culture (Caplen, Parrish et al. 2001; Elbashir, Harborth et al. 2001). These siRNAs do not appear to induce the interferon response in mammalian cells in culture. One reason for this may be that these siRNAs are too small to activate PKR.

Researchers have always been pessimistic about applying RNAi to mammalian cells because exposing such cells to dsRNA, of any sequence, triggers a global shut down of protein synthesis. Additionally, the process of effectively delivering siRNAs to mammalian cells in an animal (noninvasive transportation of the siRNA to the cell) will be difficult. (Nature, v. 411, p. 428-429, May, 2001)

### SUMMARY

In a preferred embodiment we have described a process for delivering an siRNA into a cell of a mammal to inhibit nucleic acid expression. Our process comprises making siRNA consisting of a sequence that is complementary to a nucleic acid sequence to be expressed in the mammal. Then we insert the siRNA into a vessel in the mammal where the vessel fluid moves the siRNA and delivers it to the parenchymal cell where nucleic acid expression is inhibited by the siRNA.

In another preferred embodiment, we describe a process for delivering siRNA to a cell in a mammal to inhibit nucleic acid expression. The process consists of inserting the siRNA into a vessel, then increasing volume in the mammal to facilitate delivery. The siRNA is moved with the increased volume to where it is delivered to the cell where it inhibits nucleic acid expression.

### DETAILED DESCRIPTION

We have found that an intravascular route of administration allows a polynucleotide to be delivered to a parenchymal cell in a more even distribution than direct parenchymal injections. The efficiency of polynucleotide delivery and expression may be increased by increasing the permeability of the tissue's blood vessel. Permeability is increased by increasing the intravascular hydrostatic (physical) pressure, delivering the injection fluid rapidly (injecting the injection fluid rapidly), using a large injection volume, and increasing permeability of the vessel wall.

The term intravascular refers to an intravascular route of administration that enables a polynucleotide to be delivered to cells. Intravascular herein means within an internal tubular structure called a vessel that is connected to a tissue or organ within the body of an animal, including mammals. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein.

Afferent blood vessels of organs are defined as vessels in which blood flows toward the organ or tissue under normal physiologic conditions. Efferent blood vessels are defined as vessels in which blood flows away from the organ or tissue under normal physiologic conditions. In the heart, afferent vessels are known as coronary arteries, while efferent vessels are referred to as coronary veins.

Volume means the amount of space that is enclosed within an object or solid shape such as an organ.

Parenchymal Cells

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the polynucleotide within the tail vein of a rodent such as a mouse.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

Polynucleotides

The term nucleic acid is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. The term nucleic acids-refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

Double-stranded RNA that is responsible for inducing RNAi is termed interfering RNA. The term siRNA means short interfering RNA which is double-stranded RNA that is less than 30 bases and preferably 21-25 bases in length.

A polynucleotide can be delivered to a cell to express an exogenous nucleotide sequence, to inhibit, eliminate, augment, or alter expression of an endogenous nucleotide sequence, or to express a specific physiological characteristic not naturally associated with the cell. Polynucleotides may be anti-sense.

We demonstrate that delivery of siRNA to cells of post-embryonic mice and rats interferes with specific gene expression in those cells. The inhibition is gene specific and does not cause general translational arrest. Thus RNAi can be effective in post-embryonic mammalian cells in vivo.

Permeability

In another preferred embodiment, the permeability of the vessel is increased. Efficiency of polynucleotide delivery and expression was increased by increasing the permeability of a blood vessel within the target tissue. Permeability is defined here as the propensity for macromolecules such as polynucleotides to move through vessel walls and enter the extravascular space. One measure of permeability is the rate at which macromolecules move through the vessel wall and out of the vessel. Another measure of permeability is the lack of force that resists the movement of polynucleotides being delivered to leave the intravascular space.

To obstruct, in this specification, is to block or inhibit inflow or outflow of blood in a vessel. Rapid injection may be combined with obstructing the outflow to increase permeability. For example, an afferent vessel supplying an organ is rapidly injected and the efferent vessel draining the tissue is ligated transiently. The efferent vessel (also called the venous outflow or tract) draining outflow from the tissue is also partially or totally clamped for a period of time sufficient to allow delivery of a polynucleotide. In the reverse, an efferent is injected and an afferent vessel is occluded.

In another preferred embodiment, the intravascular pressure of a blood vessel is increased by increasing the osmotic pressure within the blood vessel. Typically, hypertonic solutions containing salts such as NaCl, sugars or polyols such as mannitol are used. Hypertonic means that the osmolarity of the injection solution is greater than physiologic osmolarity. Isotonic means that the osmolarity of the injection solution is the same as the physiological osmolarity (the tonicity or osmotic pressure of the solution is similar to that of blood). Hypertonic solutions have increased tonicity and osmotic pressure similar to the osmotic pressure of blood and cause cells to shrink.

In another preferred embodiment, the permeability of the blood vessel can also be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Biologically-active molecules include vascular permeability factor (VPF) which is also known as vascular endothelial growth factor (VEGF). Another type of biologically-active molecule can also increase permeability by changing the extracellular connective material. For example, an enzyme could digest the extracellular material and increase the number and size of the holes of the connective material.

In another embodiment a non-viral vector along with a polynucleotide is intravascularly injected in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volumes in terms of ml/body weight can be 0.03 ml/g to 0.1 ml/g or greater.

The injection volume can also be related to the target tissue. For example, delivery of a non-viral vector with a polynucleotide to a limb can be aided by injecting a volume greater than 5 ml per rat limb or greater than 70 ml for a primate. The injection volumes in terms of ml/limb muscle are usually within the range of 0.6 to 1.8 ml/g of muscle but can be greater. In another example, delivery of a polynucleotide to liver in mice can be aided by injecting the non-viral vector—polynucleotide in an injection volume from 0.6 to 1.8 ml/g of liver or greater. In another preferred embodiment, delivering a polynucleotide—non-viral vector to a limb of a primate (rhesus monkey), the complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel to be injected into, and the size of the animal. In one embodiment the total injection volume (1-3 mls) can be injected from 15 to 5 seconds into the vascular system of mice. In another embodiment the total injection volume (6-35 mls) can be injected into the vascular system of rats from 20 to 7 seconds. In another embodiment the total injection volume (80-200 mls) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than ml per gram (target tissue weight) per second are used for gene delivery to target organs. In another embodiment injection rates of less than 0.06 ml per gram (target tissue weight) per second are used for gene delivery into limb muscle and other muscles of primates.

Reporter Molecules

There are three types of reporter (marker) gene products that are expressed from reporter genes. The reporter gene/protein systems include:

a) Intracellular gene products such as luciferase, β-galactosidase, or chloramphenicol acetyl transferase. Typically, they are enzymes whose enzymatic activity can be easily measured.

b) Intracellular gene products such as β-galactosidase or green fluorescent protein which identify cells expressing the reporter gene. On the basis of the intensity of cellular staining, these reporter gene products also yield qualitative information concerning the amount of foreign protein produced per cell.

c) Secreted gene products such as secreted alkaline phosphatase (SEAP), growth hormone, factor IX, or alphal-antitrypsin are useful for determining the amount of a secreted protein that a gene transfer procedure can produce. The reporter gene product can be assayed in a small amount of blood.

In a preferred embodiment, we provide a process for inhibiting gene expression in post-embryonic mammalian cells in vivo by delivering to a mammalian cell a siRNA comprising a double-stranded structure having a nucleotide sequence substantially identical to a sequence contained within the target gene and verifying the inhibition of expression of the target gene.

We also provide a process for delivery of siRNA to the cells of post-embryonic mammals. Specifically, this method is pressurized intravascular injection of siRNA, which are delivered to cells in vivo.

Additionally, another preferred embodiment provides a process for the delivery of siRNA to the cells of post-embryonic mammals. Specifically, this method is delivery of nucleic acids to cells via bile duct injection.

Yet another preferred embodiment provides for delivery of siRNA to the cells of post-embryonic mammals to muscle cells via pressurized injection of the iliac artery.

### EXAMPLES

The following examples are provided in order to demonstrate and further illustrate certain preferred embodiments and aspects of the present invention and are not to be construed as limiting the scope thereof.

### Example 1

Inhibition of Luciferase Gene Expression by siRNA in Liver Cells in vivo.

A. Preparation of siRNA

Single-stranded, gene-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the luc+ gene has the sequence:

5′-rCrUrUrArCrGrCrUrGrArGrUrArCrUrUrCrGrATT-3′

and corresponds to positions 155-173 of the luc+ reading frame. The letter “r” preceding a nucleotide indicates that nucleotide is a ribonucleotide.

The antisense oligomer with identity to the luc+ gene has the sequence:

5′-rUrCrGrArArGrUrArCrUrCrArGrCrGrUrArArGTT-3′

and corresponds to positions 155-173 of the luc+ reading frame in the antisense direction. The letter “r” preceding a nucleotide indicates that nucleotide is a ribonucleotide.

The annealed oligomers containing luc+ coding sequence are referred to as siRNA-luc+.

The sense oligomer with identity to the ColE1 replication origin of bacterial plasmids has the sequence:

5′-rGrCrGrArUrArArGrUrCrGrUrGrUrCrUrUrArCTT-3′

The letter “r” preceding a nucleotide indicates that nucleotide is a ribonucleotide.

The antisense oligomer with identity to the ColE1 origin of bacterial plasmids has the sequence:

5′-rGrUrArArGrArCrArCrGrArCrUrUrArUrCrGrCTT-3′

The letter “r” preceding a nucleotide indicates that nucleotide is a ribonucleotide.

The annealed oligomers containing ColE1 sequence are referred to as siRNA-ori.

B. Delivery of Target DNA and siRNA to Liver Cells in Mice

Plasmid pMIR48 (10 μg), containing the luc+ coding region (Promega Corp.) and a chimeric intron downstream of the cytomegalovirus major immediate-early enhancer/promoter, is mixed with 0.5 or 5 μg of siRNA-luc+ and diluted in 1-3 mls Ringer's solution (147 mM NaCl, 4 mM KCl, 1.13 mM CaCl₂) and injected in the tail vein over 7-120 seconds.

C. Assay of Luc+ Activity and Assessment of siRNA Induction of RNAi

One day after injection, the livers are harvested and homogenized in lysis buffer (0.1% Triton X-100, 0.1M K-phosphate, 1 mM DTT, pH 7.8). Insoluble material is cleared by centrifugation. 10 μl of the cellular extract or extract diluted 10×is analyzed for luciferase activity using the Enhanced Luciferase Assay kit (Mirus).

Co-injection of 10 μg of pMIR48 and 0.5 μg of siRNA—luc+ results in 69% inhibition of Luc+ activity as compared to injection of 10 g of pMIR48 alone. Co-injection of 5 μg of siRNA—luc+with 10 μg of pMIR48 results in 93% inhibition of Luc+ activity.

### Example 2

Inhibition of Luciferase Expression by siRNA is Gene Specific in Liver in vivo.

In this example, two plasmids are injected simultaneously with or without siRNA-luc+as described in Example 1. The first, pMIR116, contains the luc+coding region OIC intron under transcriptional control of the simian virus 40 enhancer and early promoter region. The second, pMIR122, contains the coding region for the Renilla reniformis luciferase under transcriptional control of the Simion virus 40 enhancer and early promoter region.

10 μg of pMIR116 and 1 μg of pMIR122 is injected as described in Example 1 without siRNA, or 0.5 or 5.0 μg siRNA-luc+. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the Oug siRNA-Luc+ control. siRNA-luc+ specifically inhibited the target Luc+ expression 73% at 0.5 μg co-injected siRNA-luc+ and 82% at 5.0 μg co-injected siRNA-luc+.

### Example 3

Inhibition of Luciferase Expression by siRNA is Gene Specific and siRNA Specific in Liver in vivo.

In this Example, 10 μg of pMIR116 and 1 μg of pMIR122 is injected as described in Example 1 with 5.0 μg siRNA-luc+ or 5.0 siRNA-ori. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 93% compared to siRNA-ori indicating inhibition by siRNAs is sequence specific in this organ.

### Example 4

Inhibition of Luciferase Expression by siRNA is Gene Specific and siRNA Specific in Spleen in vivo.

In this Example, 10 μg of pMIR116 and 1 μg of pMIR122 is injected as described in Example 1 with 5.0 μg siRNA-luc+ or 5.0 siRNA-ori. One day after injection, the spleens were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in spleen by 90% compared to siRNA-ori indicating inhibition by siRNAs is sequence specific in this organ.

### Example 5

Inhibition of Luciferase Expression by siRNA is Gene Specific and siRNA Specific in Lung in vivo.

In this Example, 10 μg of pMIR116 and 1 μg of pMIR122 is injected as described in Example 1 with 5.0 μg siRNA-luc+ or 5.0 siRNA-ori. One day after injection, the lungs were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in lung by 89% compared to siRNA-ori indicating inhibition by siRNAs is sequence specific in this organ.

### Example 6

Inhibition of Luciferase Expression by siRNA is Gene Specific and siRNAi Specific in Heart in vivo.

In this Example, 10 μg of pMIR116 and 1 μg of pMIR122 is injected as described in Example 1 with 5.0 μg siRNA-luc+ or 5.0 siRNA-ori. One day after injection, the hearts were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in heart by 80%.

### Example 7

Inhibition of Luciferase Expression by siRNA is Gene Specific and siRNA Specific in Kidney in vivo.

In this Example, 10 μg of pMIR116 and 1 μg of pMIR122 is injected as described in Example 1 with 5.0 μg siRNA-luc+ or 5.0 siRNA-ori. One day after injection, the kidneys were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in kidney by 90% compared to siRNA-ori indicating inhibition by siRNAs is sequence specific in this organ.

### Example 8

Inhibition of Luciferase Expression by siRNA is Gene Specific and siRNA Specific in Liver After Bile Duct Delivery in vivo.

In this example, 10 μg of pMIR116 and 1 μg of pMIR122 with 5.0 μg siRNA-luc+ or 5.0 siRNA-ori are injected into the bile duct of mice in a total volume of 1 ml in Ringer's buffer delivered at 6 ml/min. The inferior vena cava is clamped above and below the liver before injection are left on for two minutes after injection. One day after injection, the liver is harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in kidney by 88% compared to the control siRNA-ori.

### Example 9

Inhibition of Luciferase Expression by siRNA is Gene Specific and siRNA Specific in Muscle in vivo After Intravascular Delivery.

In this example, 10 μg of pMIR116 and 1 μg of pMIR122 with 5.0 μg siRNA-luc+ or 5.0 siRNA-ori were injected into iliac artery of rats under high pressure. Specifically, animals are anesthetized and the surgical field shaved and prepped with an antiseptic. The animals are placed on a heating pad to prevent the loss of body heat during the surgical procedure. A midline abdominal incision will be made after which skin flaps will be folded away with clamps to expose the target area. A moist gauze will be applied to prevent excessive drying of internal organs. Intestines will be moved to visualize the iliac veins and arteries. Microvessel clips are placed on the external iliac, caudal epigastric, internal iliac, deferent duct, and gluteal arteries and veins to block both outflow and inflow of the blood to the leg. An efflux enhancer solution (e.g., 0.5 mg papaverine in 3 ml saline) is injected into the external iliac artery though a 25-27 g needle, followed by the plasmid DNA and siRNA containing solution (in 10 ml saline) 1-10 minutes later. The solution is injected in approximately 10 seconds. The microvessel clips are removed 2 minutes after the injection and bleeding controlled with pressure and gel foam. The abdominal muscles and skin are closed with 4-0 dexon suture. Each procedure takes approximately 15 minutes to perform.

Four days after injection, rats were sacrificed and the quadricep and gastrocnemius muscles were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in qaudriceps and gastrocnemius by 85% and 92%, respectively, compared to the control siRNA-ori.

### Example 10

RNAi of SEAP Reporter Gene Expression Using siRNA in vivo.

Single-stranded, SEAP-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the SEAP reporter gene has the sequence:

5′-rArGrGrGrCrArArCrUrUrCrCrArGrArCrCrArUTT-3′

and corresponds to positions 362-380 of the SEAP reading frame in the sense direction. The letter “r” preceding a nucleotide indicates that nucleotide is a ribonucleotide.

The antisense oligomer with identity to the SEAP reporter gene has the sequence:

5′-rArUrGrGrUrCrUrGrGrArArGrUrUrGrCrCrCrUTT-3′

and corresponds to positions 362-380 of the SEAP reading frame in the antisense direction. The letter “r” preceding a nucleotide indicates that nucleotide is a ribonucleotide.

The annealed oligomers containing SEAP coding sequence are referred to as siRNA-SEAP.

Plasmid pMIR141 (10 μg), containing the SEAP coding region (Promega Corp.) under transcriptional control of the human ubiquitin C promoter and the human hepatic control region of the apolipoprotein E gene cluster, is mixed with 0.5 or 5 μg of siRNA-SEAP or 5 μg siRNA-ori and diluted in 1-3 mls Ringer's solution (147 mM NaCl, 4 mM KCl, 1.13 mM CaCl₂) and injected in the tail vein over 7-120 seconds. Control mice also include those injected with pMIR141 alone.

Each mouse is bled from the retro-orbital sinus one day after injection. Cells and clotting factors are pelleted from the blood to obtain serum. The serum is evaluated for the presence of SEAP by a chemiluminescence assay using the Tropix Phospha-Light kit.

Results indicate SEAP expression was inhibited by 59% when 0.5 μg siRNA-SEAP was delivered and 83% when 5.0 μg siRNA-SEAP was delivered. No decrease in SEAP expression was observed when 5.0 μg of siRNA-ori was delivered indicating the decrease in SEAP expression by siRNA-SEAP is gene specific.

Day 1

| | AVE SEAP (ng/ml) | SD |
|---|---|---|
| plasmid only | 2239 | 1400 |
| siRNA-ori (50 μg) | 2897 | 1384 |
| siRNA-SEAP (0.5 μg) | 918 | 650 |
| siRNA-SEAP (5.0 μg) | 384 | 160 |

### Example 11

Inhibition of Endogenous Mouse Cytosolic Alanine Aminotransferase (ALT) Expression after in vivo Delivery of siRNA.

Single-stranded, cytosolic alanine aminotrasferase-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the endogenous mouse and rat gene encoding cytosolic alanine aminotransferase has the sequence:

5′-rCrArCrUrCrArGrUrCrUrCrUrArArGrGrGrCrUTT-3′

and corresponds to positions 928-946 of the cytosolic alanine aminotransferase reading frame in the sense direction. The letter “r” preceding a nucleotide indicates that nucleotide is a ribonucleotide.

The sense oligomer with identity to the endogenous mouse and rat gene encoding cytosolic alanine aminotransferase has the sequence:

5′-rArGrCrCrCrUrUrArGrArGrArCrUrGrArGrUrGTT-3′

and corresponds to positions 928-946 of the cytosolic alanine aminotransferase reading frame in the antisense direction. The letter “r” preceding a nucleotide indicates that nucleotide is a ribonucleotide.

The annealed oligomers containing cytosolic alanine aminotransferase coding sequence are referred to as siRNA-ALT

Mice are injected in the tail vein over 7-120 seconds.with 40 μg of siRNA-ALT diluted in 1-3 mls Ringer's solution (147 mM NaCl, 4 mM KCl, 1.13 mM CaCl₂). Control mice were injected with Ringer's solution without siRNA. Two days after injection, the livers were harvested and homogenized in 0.25 M sucrose. ALT activity was assayed using the Sigma diagnostics INFINITY ALT reagent according to the manufacturers instructions. Total protein was determined using the BioRad Protein Assay. Mice injected with 40 μg of siRNA-ALT had a 32% average decrease in ALT specific activity compared to that of mice injected with Ringer's solution alone.

### Example 12

We have achieved expression of the LDL receptor in low-density lipoprotein receptor (LDLR) (−/−) mice, which lowers triglycerides. For these experiments, mice lacking the LDLR were used. These mice have elevated lipoprotein levels. Expression of the LDLR in the liver is expected to result in lowering of lipoproteins. To this end, 100 μg of pCMV-LDLR was injected into the bile duct of LDLR (−/−) mice (obtained form The Jackson Laboratories). Blood was obtained one day prior and one day after plasmid DNA injection and analyzed for triglycerides levels. The average triglycerides level before injection was 209±69 mg/dl. One day after pDNA delivery, triglyceride levels were measured at 59±14 mg/dl. We included a few normal mice, in which triglyceride levels were lowered as well.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1) A process for delivering an siRNA into a cell of a mammal to inhibit nucleic acid expression, comprising:
a) making siRNA consisting of a sequence that is complementary to a nucleic acid sequence to be expressed in the mammal;
b) inserting the siRNA into a vessel in the mammal;
c) delivering the siRNA to the parenchymal cell wherein the nucleic acid expression is inhibited.

2) The process of claim 1 wherein vessel permeability is increased.

3) The process of claim 2 wherein increasing the permeability of the vessel consists of increasing pressure against vessel walls.

4) The process of claim 3 wherein increasing the pressure consists of increasing a volume of fluid within the vessel.

5) The process of claim 4 wherein increasing the volume consists of inserting the siRNA in solution into the vessel.

6) The process of claim 1 wherein the vessel consists of a tail vein.

7) The process of claim 1 wherein the vessel consists of a bile duct.

8) The process of claim 1 wherein the parenchymal cell is a cell selected from the group consisting of liver cells, spleen cells, heart cells, kidney cells and lung cells.

9) The process of claim 1 wherein the siRNA is injected into the mammal's vessel.

10) The process of claim 9 wherein the permeability of the vessel is increased.

11) A process for delivering siRNA to a cell in a mammal to inhibit nucleic acid expression, comprising:
a) inserting the siRNA into a vessel;
b) increasing volume in the mammal to facilitate delivery;
c) delivering the siRNA to the cell; and,
d) inhibiting nucleic acid expression.

12) The process of claim 11 wherein vessel permeability is increased.

13) The process of claim 12 wherein increasing the permeability of the vessel consists of increasing pressure against vessel walls.

14) The process of claim 13 wherein increasing the pressure consists of increasing a volume within the vessel.

15) The process of claim 14 wherein the pressure is sufficient to increase organ volume.

16) The process of claim 13 wherein increasing the pressure consists of increasing a volume within the vessel.

17) The process of claim 16 wherein the pressure is sufficient to increase extravascular volume.

18) The process of claim 11 wherein the siRNA is complementary to a mammalian nucleic acid sequence.

19) The process of claim 11 wherein the siRNA is complementary to a viral nucleic acid sequence.
